(19) United States
(12) Patent Application Publication
(10) Pub. No.: US 2017/0128608 A1
(43) Pub. Date: May 11, 2017

(54) AROMATIC NEBULIZING DIFFUSER HAVING A REPLACEABLE OUTER HOUSING

(71) Applicant: Ming Jen Hsiao, Miaoli County (TW)

(72) Inventor: Ming Jen Hsiao, Miaoli County (TW)

(21) Appl. No.: 14/938,564

(22) Filed: Nov. 11, 2015

(51) Int. Cl. A61L 9/03; H05B 3/00

(52) U.S. Cl. CPC A61L 9/03; H05B 3/0014

(57) ABSTRACT

An aromatic nebulizing diffuser includes an inner housing, an outer housing having an outer surface area of diamond cut facets and a center hole by which the outer housing is detachably mounted around the inner housing, a thermally conductive container mounted in the inner housing, an electric heating element mounted at the bottom side of the thermally conductive container, and an angle-adjustable electrical plug electrically coupled with the electric heating element for connection to an external power outlet and allowing adjustment of the angular position of the inner housing and the outer housing relative to the angle-adjustable electrical plug to the desired angle.

### BACKGROUND OF THE INVENTION

1. Field of the Invention: The present invention relates to an aromatic nebulizing diffuser and more particularly, to such an aromatic nebulizing diffuser, which facilitates replacement of a different design of outer housing.

2. Description of the Related Art:

Various conventional aromatic nebulizing diffusers are not all suitable for use in cars. Even if they are designed for use in a car and electrically connectable to a car cigarette lighter receptacle, their installation angle is not adjustable. If an aromatic nebulizing diffuser is connected to a car cigarette lighter receptacle, the contained aromatic substance must be kept to face upward to avoid flowing of the melted aromatic substance out of the aromatic substance container to contaminate the surroundings. Although there are angle-adjustable aromatic nebulizing diffusers, however, the angle adjustment structures of these angle-adjustable aromatic nebulizing diffusers are commonly complicated, resulting in a high cost. Further, these angle adjustment structures wear quickly with use.

Further, commercial aromatic nebulizing diffusers are commonly monotonous in shape design, and not allowed for change of the design of the outer housing to provide enhanced decorative effects.

Therefore, conventional aromatic nebulizing diffusers still have room for improvement.

### SUMMARY OF THE INVENTION

The present invention has been accomplished under the circumstances in view. It is main object of the present invention to provide an aromatic nebulizing diffuser, which allows change of the outer housing, providing enhanced decorative effects.

It is object of the present invention to provide an aromatic nebulizing diffuser, which facilitates adjustment of the device use angle, achieving a high level of application safety.

To achieve these and other objects of the present invention, an aromatic nebulizing diffuser comprises an inner housing comprising a top opening and a mounting member, an outer housing detachably mounted around the inner housing and comprising a center hole adapted for receiving the inner housing, a thermally conductive container mounted inside the inner housing and having a top open side facing toward the top opening of the inner housing, an electric heating element mounted at a bottom side of the thermally conductive container and adapted for heating the thermally conductive container, and an angle-adjustable electrical plug device fastened to the mounting member of the inner housing and electrically coupled with the electric heating element and adapted for connection to an external power source.

Further, the outer housing has an outer surface area of diamond cut facets to provide enhanced decorative effects. By means of the design of the center opening, the outer housing is detachably mountable around the inner housing. Thus, different designs of outer housing can be provided and selected for mounting around the inner housing in a replaceable manner to exhibit a decorative effect.

Further, the angle-adjustable electrical plug device is configured for connection to an external power outlet, for example, the angle-adjustable electrical plug device can be made in the form of a car cigarette lighter for connection to a car cigarette lighter receptacle. After insertion of the angle-adjustable electrical plug device of the aromatic nebulizing diffuser into the car cigarette lighter receptacle of a car, the outer housing can be biased with the inner housing relative to the angle-adjustable electrical plug device to let the top opening of the inner housing, the center hole of the outer housing and the top open side of the thermally conductive container face upward, preventing falling of the aromatic substance out of the thermally conductive container to contaminate the instrument board of the car or other surroundings. Further, an aroma capsule can be put in the thermally conductive container. After the electric heating element is electrically conducted, it generates heat that is then transferred upwardly through the thermally conductive container to heat the aroma capsule, causing it to release a pleasant smell.

Other advantages and features of the present invention will be fully understood by reference to the following specification in conjunction with the accompanying drawings, in which like reference signs denote like components of structure.

### BRIEF DESCRIPTION OF THE DRAWINGS

FIG. 1 is an elevational view of an aromatic nebulizing diffuser in accordance with the present invention.

FIG. 2 is an exploded view of the aromatic nebulizing diffuser in accordance with the present invention (I).

FIG. 3 is an exploded view of the aromatic nebulizing diffuser in accordance with the present invention (II).

FIG. 4 is a longitudinal sectional view of the aromatic nebulizing diffuser in accordance with the present invention.

FIG. 5 is a side view of the aromatic nebulizing diffuser in accordance with the present invention.

FIG. 6 is a bottom view of the aromatic nebulizing diffuser in accordance with the present invention.

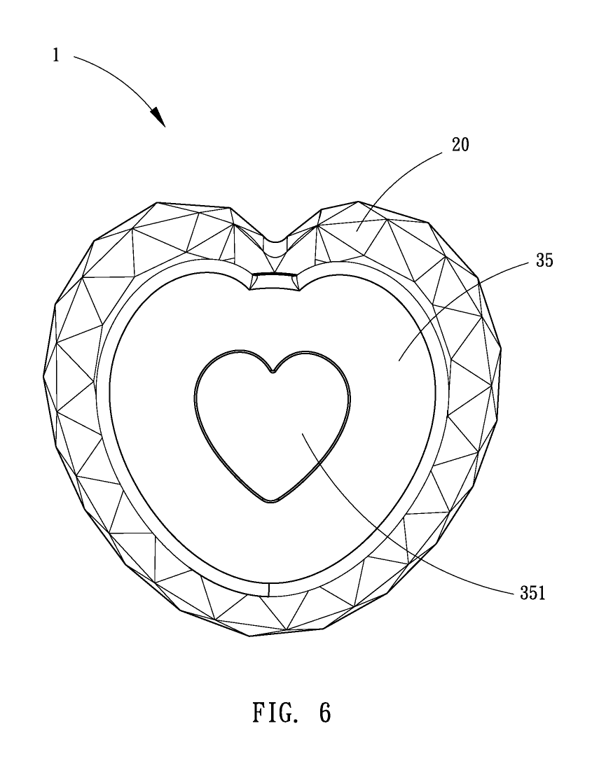

### DETAILED DESCRIPTION OF THE PREFERRED EMBODIMENT

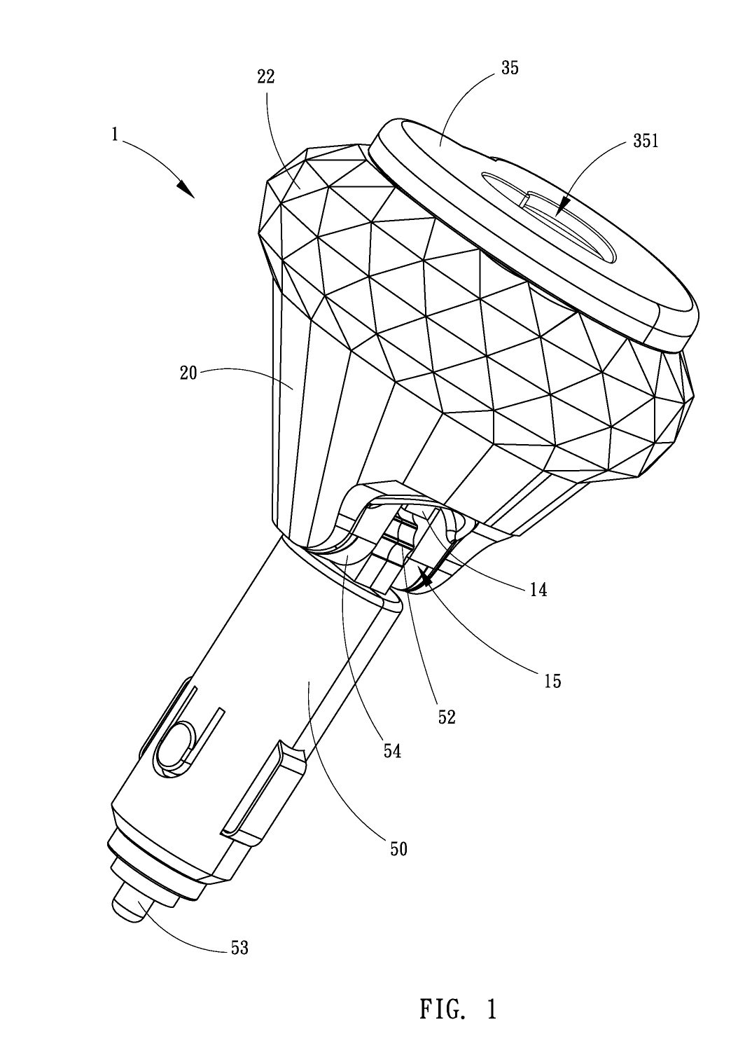

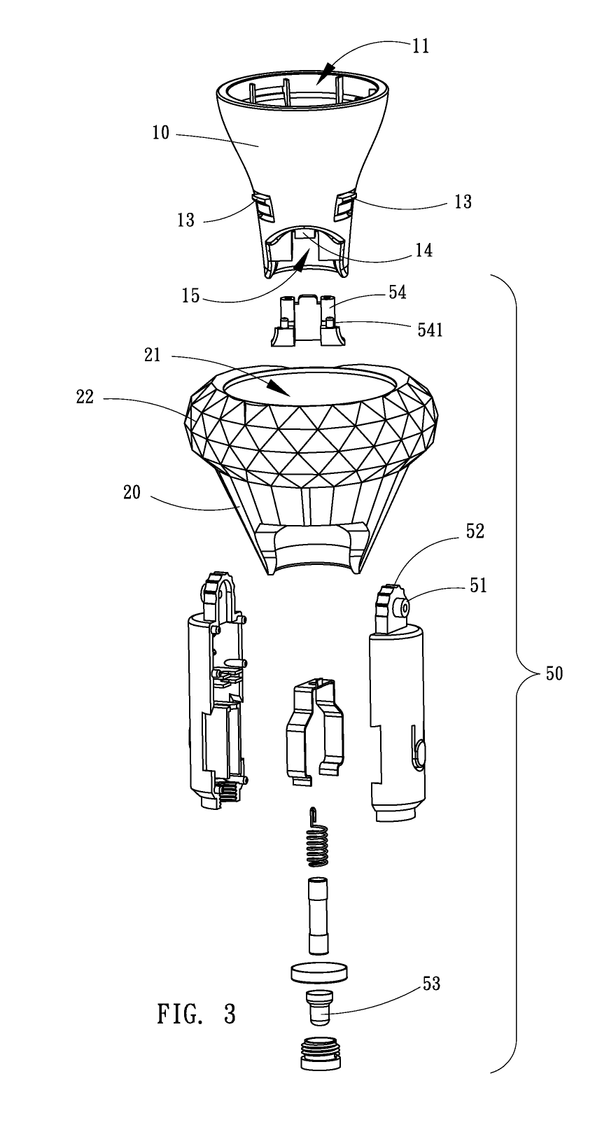

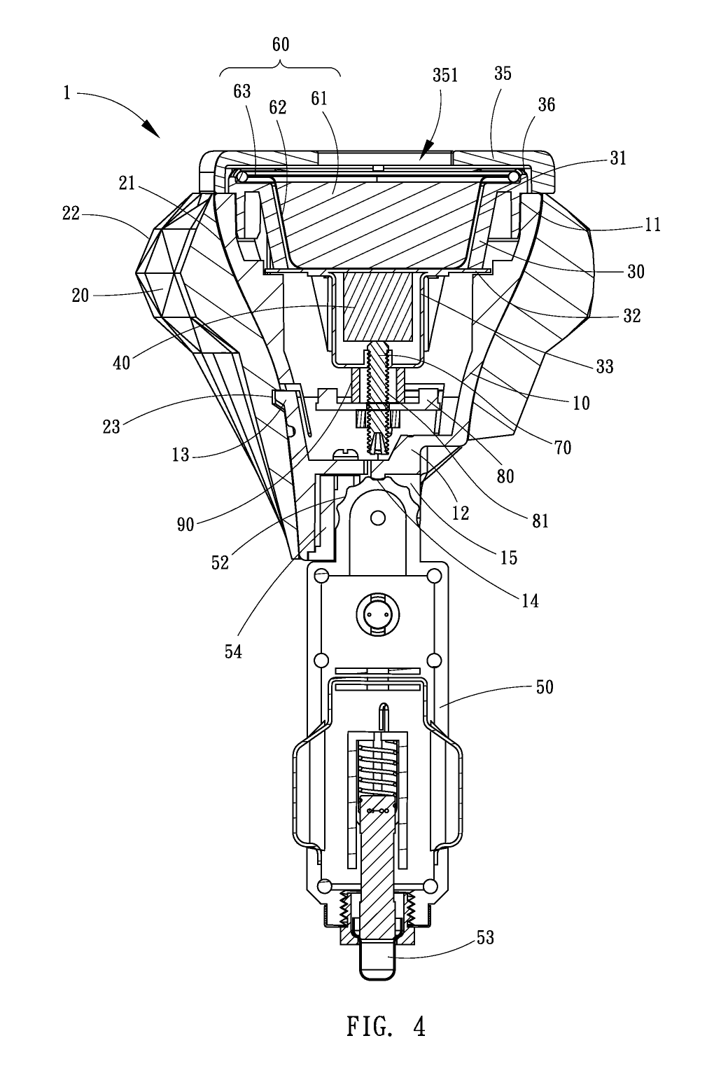

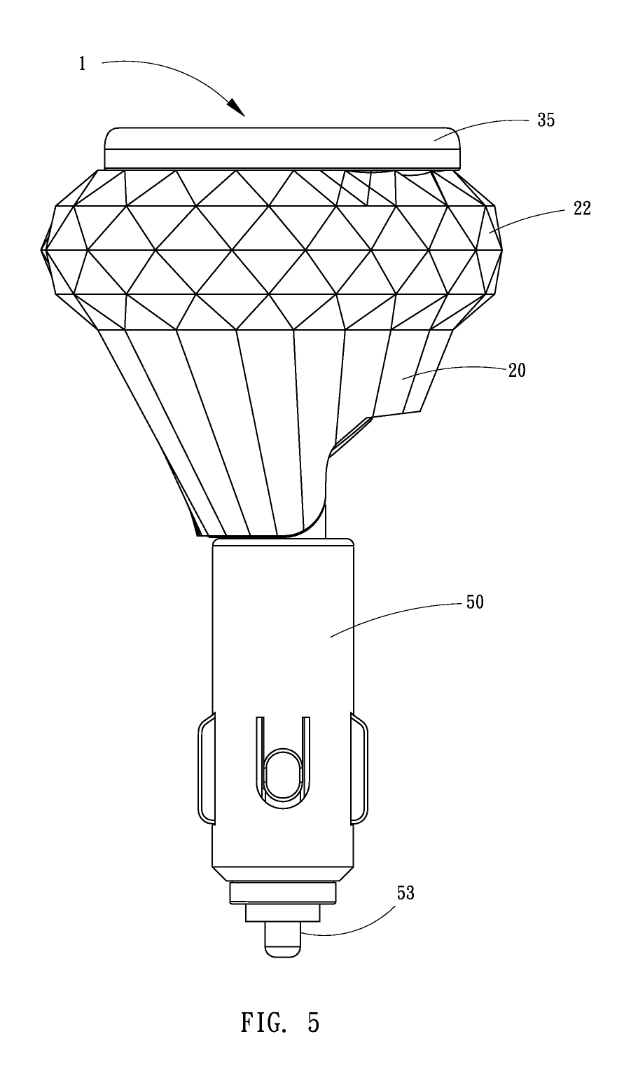

Referring to FIGS. 1-5, an aromatic nebulizing diffuser 1 in accordance with the present invention is shown. The aromatic nebulizing diffuser 1 comprises an inner housing 10 comprising a top opening 11 and a mounting member 12, an outer housing 20 comprising a center hole 21 that receives the inner housing 10, a thermally conductive container 30 mounted inside the inner housing 10 and having a top open side 31 facing toward the top opening 11 of the inner housing 10, an electric heating element 40 fastened to a bottom wall of the thermally conductive container 30, and an angle-adjustable electrical plug device 50 fastened to the mounting member 12 of the inner housing 10 and electrically coupled with the electric heating element 40.

In this embodiment, the outer housing 20 has an outer surface area of diamond cut facets 22.

Based on the architecture described above, the features, advantages of the aromatic nebulizing diffuser 1 are outlined hereinafter.

The outer housing 20 comprises, but not limited to, the aforesaid outer surface area of diamond cut facets 22. By means of the center hole 21, the outer housing 20 is detachably mounted around the inner housing 10. Thus, different designs of outer housing 20 can be provided and selected for mounting around the inner housing 10 in a replaceable manner to exhibit a decorative effect.

The inner housing 10 and the angle-adjustable electrical plug device 50 can be biased relative to each other to adjust the installation angle, allowing the angle-adjustable electrical plug device 50 to be conveniently connected to a power outlet (not shown). After installation, the angular position of the inner housing 10 relative to the angle-adjustable electrical plug device 50 can be adjusted, keeping the top opening 11 of the inner housing 10, the center hole 21 of the outer housing 20 and the top open side 31 of the thermally conductive container 30 to face upward. Further, the aromatic nebulizing diffuser 1 can be adjusted to bias the thermally conductive container 30 from an upright position to an inclined position, minimizing space occupation.

Further, in this embodiment, the angle-adjustable electrical plug device 50 of the aromatic nebulizing diffuser 1 is made in the form of a car cigarette lighter for connection to a car cigarette lighter receptacle. After insertion of the angle-adjustable electrical plug device 50 of the aromatic nebulizing diffuser 1 into the car cigarette lighter receptacle of a car, the outer housing 20 can be biased with the inner housing 10 relative to the angle-adjustable electrical plug device 50 to let the top opening 11 of the inner housing 10, the center hole 21 of the outer housing 20 and the top open side 31 of the thermally conductive container 30 face upward. Further, an aroma capsule 60 can be put in the thermally conductive container 30 through the top open side 31. After the electric heating element 40 is electrically conducted, it generates heat that is then transferred upwardly through the thermally conductive container 30 to heat the aroma capsule 60, causing it to release a pleasant smell.

Referring to FIGS. 1-4 again, the inner housing 10 comprises a plurality of male retaining members 13 spaced around the periphery thereof. The outer housing 20 comprises a plurality of female retaining members, for example, retaining grooves 23 spaced around an inside wall thereof in the center hole 21 and respectively forced into engagement with the respective male retaining members 13 to secure the outer housing 20 to the inner housing 10.

The male retaining members 13 can be, for example, hooks, protruding blocks or fasteners. In this embodiment, the male retaining members 13 are elastically deformable hooks. The male retaining members 13 can be pressed (by the inside wall of the outer housing 20 around the center hole 21) and disengaged from the respective retaining grooves 23. When the external force is disappeared, the male retaining members 13 immediately return to their former shape and forced into engagement with the respective retaining grooves 23, thus, the outer housing 20 can conveniently be attached to the periphery of the inner housing 10 and detachably secured thereof, facilitating replacement.

Referring to FIGS. 1-4 again, the angle-adjustable electrical plug device 50 comprises a pivot rod 51 transversely disposed at one end thereof, a ratchet 52 located on a middle part of the pivot rod 51, an electrically conducting terminal 53 located at an opposite end thereof remote from the pivot rod 51, and a pivot holder 54. The pivot holder 54 comprises two pivot holes 541 transversely aligned in line. The inner housing 10 further comprises a damping block 14 disposed therein, and a side opening 15 cut through the peripheral wall thereof. The pivot holder 54 is fastened to the mounting member 12 of the inner housing 10. The pivot rod 51 of the angle-adjustable electrical plug device 50 is coupled to the pivot holes 541 of the pivot holder 54 to abut the ratchet 52 against the damping block 14, allowing the angle-adjustable electrical plug device 50 to be biased relative to the inner housing 10 within the side opening 15 and then positioned in one of a series of angles. The electrically conducting terminal 53 is electrically connectable to an electric outlet, for example, a car cigarette lighter receptacle. Subject engagement between the ratchet 52 of the angle-adjustable electrical plug device 50 and the damping block 14 of the inner housing 10, the inner housing 10, outer housing 20 and thermally conductive container 30 of the aromatic nebulizing diffuser 1 can be biased relative to the angle-adjustable electrical plug device 50 to the desired angular position within a limited range, and thus, after connection of the angle-adjustable electrical plug device 50 to an electric outlet, for example, a car cigarette lighter receptacle, the user can bias the inner housing 10, outer housing 20 and thermally conductive container 30 of the aromatic nebulizing diffuser 1 relative to the angle-adjustable electrical plug device 50 to such an angular position where the top opening 11 of the inner housing 10 and the top open side 31 of the thermally conductive container 30 face upward, and then put an aroma capsule 60 into the thermally conductive container 30, enabling the aroma capsule 60 to be heated to release a pleasant smell to the atmosphere through the top open side 31 of the thermally conductive container 30 and the top opening 11 of the inner housing 10. Preferably, the side opening 15 is configured for enabling the angle-adjustable electrical plug device 50 to be biased relative to the inner housing 10 within 90° angle, preventing the ratchet 52 from being jammed in the pivot holder 54 and the side opening 15 of the inner housing 10 and the thermally conductive container 30 with the aroma capsule 60 from being excessively tilted.

In the present preferred embodiment, the thermally conductive container 30 further comprises a heat transfer plate 32 bonded to the bottom wall thereof.

The thermally conductive container 30 further comprises a bracket 33 located at a bottom side of the heat transfer plate 32. The electric heating element 40 is abutted against a bottom surface of the heat transfer plate 32, and disposed inside the bracket 33. The aromatic nebulizing diffuser 1 further comprises a screw rod 70 threaded into the bracket 33 and stopped at the electric heating element 40 against the heat transfer plate 32 of the thermally conductive container 30 to keep the electric heating element 40 in positive contact with the heat transfer plate 32.

In this embodiment, the peripheral wall of the thermally conductive container 30 can be made by a thermally insulative material; the heat transfer plate 32 is selected from the group of metal materials and their alloys. Thus, heat generated by the electric heating element 40 can simply be transferred to the heat transfer plate 32 and the bottom wall of the thermally conductive container 30 to heat a bottom surface of the aroma capsule 60 evenly, avoiding concentration of heat energy on the center of the bottom surface of the aroma capsule 60 and prolonging the effective working time of the aroma capsule 60. Further, as an alternate form of the present invention, the bottom wall of the thermally conductive container 30 can be formed of the heat transfer plate 32. Because the peripheral wall of the thermally conductive container 30 is thermally nonconductive, it prohibits transfer of heat energy to the inner housing 10 and the outer housing 20.

In the present preferred embodiment, the aromatic nebulizing diffuser 1 further comprises a PC board 80 fixedly mounted inside the inner housing 10 and electrically coupled with the angle-adjustable electrical plug device 50 and the electric heating element 40. Further, the PC board 80 comprises a screw hole 81. The screw rod 70 is threaded through the screw hole 81 with one end thereof fastened to the bracket 33 and stopped against the electric heating element 40. Further, a thermally insulative spacer 90 is mounted around the screw rod 70 and stopped between the bracket 33 and the PC board 80 to prohibit direct transfer of heat energy from the electric heating element 40 to the PC board 80, enhancing stability and reliability of the PC board 80.

The electric heating element 40 can be, for example, a resistor, a positive temperature coefficient (PTC) thermistor, cement resistor or Mica heating element. In this embodiment, the electric heating element 40 is a semiconductor ceramic heating element of positive temperature coefficient (PTC) thermistor for the advantage of high thermal stability practical for heating aromatic substances.

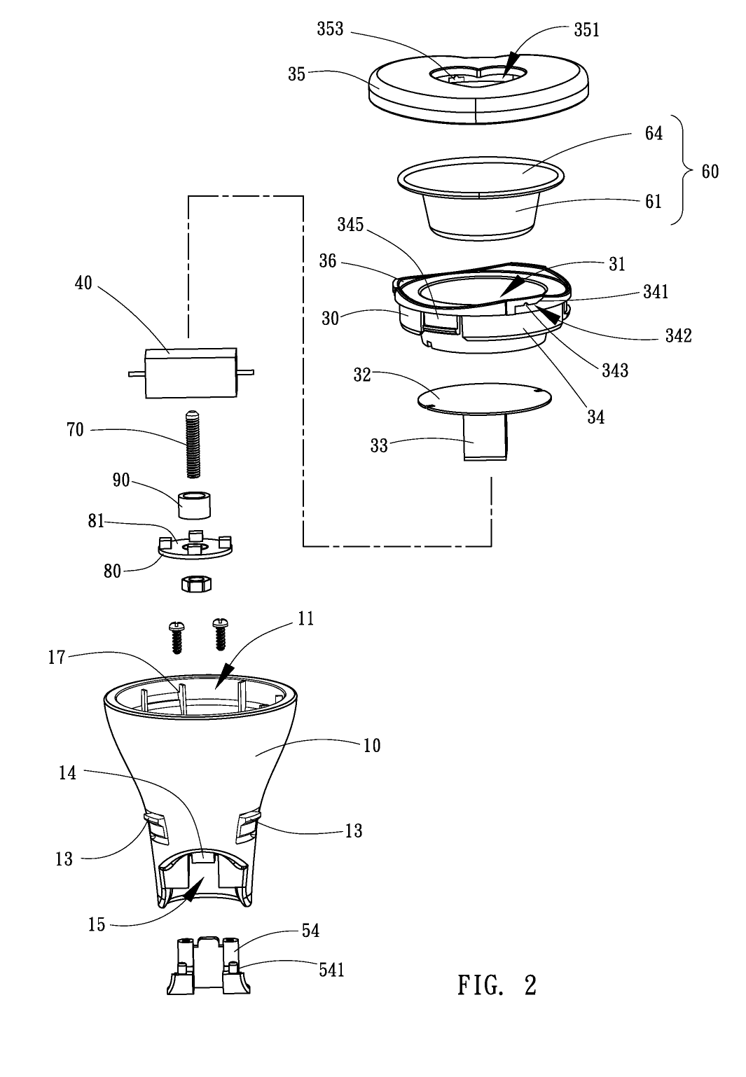

As illustrated in FIGS. 2, 3 and 4, the thermally conductive container 30 further comprises a plurality of positioning recesses 34 spaced around the periphery thereof. Each positioning recess 34 comprises a sloping surface portion 341, a recessed portion 342 located at one end of the sloping surface portion 341, and an engagement notch 343 located at a top side of the recessed portion 342. The aromatic nebulizing diffuse 1 further comprises a container cap 35 capped on the thermally conductive container 30 over the top open side 31. The container cap 35 comprises a center opening 351, and a plurality of engagement protrusions 353 spaced around the inside wall thereof corresponding to the positioning recesses 34 of the thermally conductive container 30 for engaging into the respective engagement notches 343 to detachably secure the container cap 35 to the thermally conductive container 30 for easy replacement of the aroma capsule 60. Further, the center opening 351 of the container cap 35 can be configured to exhibit a heart-shaped, a petal-like shape, the shape of an animal, a geometric shape, or a shape trademark, providing enhanced decorative effects.

The thermally conductive container 30 further comprises at least one retaining member 345. In this embodiment, the retaining member 345 is a spring hook extended from the periphery of the thermally conductive container 30. The inner housing 10 further comprises at least one lug 17 for the engagement of the at least one retaining member 345 of the thermally conductive container 30 to secure the thermally conductive container 30 to the inside of the inner housing 10.

Referring to FIGS. 2-4 again, the configuration of the center opening 351 and contour of the container cap 35 exhibits a heart-shaped configuration. When the aroma capsule 60 is heated, it releases a cool, dry, fragrant mist of pleasant smell that goes out of the center opening 351 to the atmosphere, producing an aromatherapy effect.

As stated above, the aroma capsule 60 is disposed in the heat-transfer container 2. According to the present preferred embodiment, the aroma capsule 60 comprises a disposable capsule body 61 and an aromatic substance 62. The disposable capsule body 61 has a top opening. The aromatic substance 62 is disposed in the disposable capsule body 61. The aromatic substance 62 is an aromatic substance block, for example. The aromatic substance block is a solid aromatic wax and essential oil, for example. In an embodiment, the aromatic substance block is an aromatic wax. Therefore, the heating component 40, when being electrically connected to a power supply, will generate heat to the heat-transfer container 30. The heat-transfer plate 32 transfers the heat to the disposable capsule body 61 and the aromatic substance 62, for the aromatic substance 62 to generate fragrance.

The aroma capsule 60 further comprises a breathing film 63 sealed to the top open side of the disposable capsule body 61.

The breathing film 63 has tiny open spaces therein that diffuse the fragrance of the aromatic substance 62 upward to an ambient environment. The breathing film 63 is a cloth (e.g., non-woven fabric, fiber cloth, linen, and canvas), fiber sheet, perforation film or perforation metal foil. In an embodiment, the breathing film 63 is made of a perforation film. The perforation film has its periphery mounted to the top opening of the disposable capsule body 61. If the angle-adjustable aroma diffuser 1 is toppled and fell accidentally, the essential oil or fluid aroma wax of the aromatic substance 62 in the disposable capsule body 61 will be blocked by the breathing film 63 from flowing in a car or another usage space. Therefore, the inner space of the car will not be contaminated, and the angle-adjustable aroma diffuser 1 is safe to use.

The aroma capsule 60 can be made with or without the breathing film 63. The aroma capsule 60 further comprises a sealing cover 64 sealed to the disposable capsule body 61 over the top open side thereof to protect the aromatic substance 62 (e.g., aroma wax) in the aroma capsule 60 from being contaminated or losing its fragrance. In use, the sealing cover 64 is opened from the top open side of the disposable capsule body 61, and then the aroma capsule 60 is put in the heat-transfer container 30.

Further, the disposable capsule body 61 can be selectively made of metal, hard plastic, fiber bowl (plant fibers, such as corn fiber, glass fiber and carbon fiber), or composition material.

In one embodiment of the present invention, the disposable capsule body 61 is an aluminum foil bowl made of an aluminum foil. Therefore, the disposable capsule body 61 is thin and light, and has a well enough heat-transfer function. The disposable capsule body 61 can conduct the heat generated by the heating component 40 to the disposable capsule body 61 and the aromatic substance 62 such that the aromatic substance 62 is heated up and diffuses fragrance. Because the disposable capsule body 61 is made of aluminum foil, it is tough and not likely to be broken, eliminating the drawback of fragile products in the prior art designs, such as ceramic and glass bowl for containing essential oil or aroma wax.

Since the aromatic substance 62 is disposed in the disposable capsule body 61, rather than disposed in the conventional container, a user, when using the aromatic substance 62, is allowed to tear off the sealing cover 64, without worrying about being contaminated by the aromatic substance 62. When the aromatic substance 62 is heated up and volatile completely through the top open side 31 of the heat-transfer container 30, a user is allowed to remove the disposable capsule body 61 from the top open side 31 of the heat-transfer container 30, and place in the heat-transfer container 30 a new aroma capsule 60 that contains aromatic substance of the same or different fragrance. Since the disposable capsule body 61 has an inner housing that is made of an aluminum foil, a user does not need to worry about breaking or having to wash the disposable capsule body 61. The user does not need to prepare an additional container for the aromatic substance 62.

Further, the thermally conductive container 30 comprises a wave-like top flange 36 extending around the top open side thereof. After insertion of the aroma capsule 60 in the thermally conductive container 30, the rim of the disposable capsule body 61 of the aroma capsule 60 is secured by the top flange 36 between the thermally conductive container 30 and the container cap 35, preventing displacement of the aroma capsule 60 relative to the thermally conductive container 30. Further, through gaps between downwardly curved areas of the top flange 36 and the rim of the disposable capsule body 61, the user can remove the disposable capsule body 61 of the aroma capsule 60 from the top open side 31 of the thermally conductive container 30 with the fingers conveniently.

As stated above, the angle-adjustable electrical plug device 50 can be made in the form of a car cigarette lighter for connection to a car cigarette lighter receptacle, enabling the aromatic nebulizing diffuser 1 to diffuse a pleasant smell in the car.

Although a particular embodiment of the invention has been described in detail for purposes of illustration, various modifications and enhancements may be made without departing from the spirit and scope of the invention. Accordingly, the invention is not to be limited except as by the appended claims.

What the invention claimed is:

1. An aromatic nebulizing diffuser, comprising:
an inner housing comprising a top opening and a mounting member;
an outer housing detachably mounted around said inner housing, said outer housing comprising a center hole adapted for receiving said inner housing;
a thermally conductive container mounted inside said inner housing, said thermally conductive container defining a top open side facing toward said top opening of said inner housing;
an electric heating element mounted at a bottom side of said thermally conductive container and adapted for heating said thermally conductive container; and
an angle-adjustable electrical plug device fastened to said mounting member of said inner housing and electrically coupled with said electric heating element and adapted for connection to an external power source.

2. The aromatic nebulizing diffuser as claimed in claim 1, wherein said inner housing comprises at least one male retaining member located at the periphery thereof; said outer housing comprises at least one retaining groove located in an inside wall thereof in said center hole and detachably engaged with said at least one male retaining member.

3. The aromatic nebulizing diffuser as claimed in claim 1, wherein said thermally conductive container comprises at least one retaining member located at the periphery thereof; said inner housing further comprises at least one lug for the engagement of said at least one retaining member of said thermally conductive container to secure said thermally conductive container to the inside of said inner housing.

4. The aromatic nebulizing diffuser as claimed in claim 1, wherein said inner housing further comprises a damping block located at an inside wall thereof and a side opening cut through the periphery thereof; said angle-adjustable electrical plug device comprises a pivot rod transversely disposed at one end thereof, a ratchet located on a middle part of said pivot rod and abutted against said damping block, an electrically conducting terminal longitudinally disposed at an opposite end thereof and inserted through said side opening of said inner housing and adapted for connection to an external power outlet, and a pivot holder fixedly mounted inside said inner housing to secure said pivot rod and said electrically conducting terminal to said inner housing in a pivotable manner, said pivot holder comprising a pivot hole coupled with said pivot rod for enabling said electrically conducting terminal to be biased relative to said inner housing within said side opening within a predetermined angle.

5. The aromatic nebulizing diffuser as claimed in claim 1, wherein said thermally conductive container comprises a heat transfer plate located at a bottom side thereof, and a bracket located at a bottom side of said heat transfer plate; said electric heating element is mounted inside said bracket and disposed in contact with a bottom surface of said heat transfer plate; the aromatic nebulizing diffuser further comprises a screw rod threaded through said bracket and stopped at said electric heating element against said heat transfer plate.

6. The aromatic nebulizing diffuser as claimed in claim 5, further comprising a PC board mounted inside said inner housing and electrically coupled with said angle-adjustable electrical plug device and said electric heating element, said PC board comprising a screw hole fastened with said screw rod, and a thermally insulative spacer mounted around said screw rod and stopped between said bracket and said PC board.

7. The aromatic nebulizing diffuser as claimed in claim 1, wherein said thermally conductive container further comprises at least one positioning recess located in the periphery thereof, each said positioning recess comprising a sloping surface portion, a recessed portion located at one end of said sloping surface portion and an engagement notch disposed at a top side of said recessed portion; the aromatic nebulizing diffuser further comprises a container cap capped on said thermally conductive container, said container cap comprising a center opening disposed in communication with the top open side of said thermally conductive container and at least one engagement protrusion detachably engageable in said engagement notch of each said positioning recess of said thermally conductive container.

8. The aromatic nebulizing diffuser as claimed in claim 7, wherein said outer housing, said container cap and said center opening of said container cap exhibit a heart shape.

9. The aromatic nebulizing diffuser as claimed in claim 8, further comprising an aroma capsule put in said thermally conductive container, said aroma capsule comprising a disposable capsule body having a top opening, an aromatic substance contained in said disposable capsule body, and a breathing film sealed to said disposable capsule body over the top opening of said disposable capsule body.

10. The aromatic nebulizing diffuser as claimed in claim 9, wherein said thermally conductive container further comprises a wave-like top flange extending around the top open side thereof; said disposable capsule body of said aroma capsule has a rim thereof stopped between said wave-like top flange of said thermally conductive container and said container cap.
